(19) United States
(12) Patent Application Publication
(10) Pub. No.: US 2015/0226816 A1
(43) Pub. Date: Aug. 13, 2015

(54) Patient Communication in Magnetic Resonance Tomography

(71) Applicant: Volker Matschl, Bamberg (DE)

(72) Inventor: Volker Matschl, Bamberg (DE)

(21) Appl. No.: 14/615,755

(22) Filed: Feb. 6, 2015

(30) Foreign Application Priority Data
Feb. 7, 2014 (DE) 102014202301.7

(51) Int. Cl. G01R 33/28; G01R 33/341; A61B 5/00; A61B 5/055

(52) U.S. Cl. CPC G01R 33/283; A61B 5/0555; G01R 33/341; A61B 5/0042; A61B 5/7455

(57) ABSTRACT

A magnetic resonance tomograph has a component on which the head of a patient rests during imaging in a contact area. The magnetic resonance tomograph also has a patient communication device for communicating information to the patient. The patient communication device has a control device and also an actuator assigned to the contact area. The actuator, upon actuation with electric control signals by the control device, vibrates the surface of the contact area.

This application claims the benefit of DE 102014202301.7, filed on Feb. 7, 2014, which is hereby incorporated by reference in its entirety.

### FIELD

The invention relates to magnetic resonance tomography.

### BACKGROUND

When patients are being examined in a magnetic resonance tomograph, an exchange of information with the patient is frequently required. Patients are given instructions about holding their breath or the like. On the other hand, communication between the patient and other people may soothe anxieties and uncertainties, especially when a magnetic resonance tomograph with a relatively narrow bore is used. Transmission of acoustic information to a patient and from a patient is relatively complex however. On the one hand, hearing protection for patients, which attenuates acoustic signals, is frequently used during magnetic resonance tomography examinations. On the other hand, high magnetic fields may be attained in magnetic resonance tomography, which is why the use of sound transducers with permanent magnets is not possible. In addition, the use of long conducting structures without sheath current protection in a patient communication system may also be avoided in order to be compatible with a magnetic resonance tomograph.

Because of these factors, a compressed-air hose is typically used for communication with the patient. Sound waves are transmitted through the hose via the medium of air to a passive headset. However, these types of compressed-air hoses are frequently perceived as disruptive by patients and/or by operators.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the disclosed embodiments may provide a magnetic resonance tomograph with a component on which the head of a patient rests in a contact area during imaging. The disclosed embodiments may also provide a patient communication device for communicating information to the patient.

The patient communication device includes a control device and also an actuator assigned to the contact area. The actuator, on activation with electric control signals by the control device, imparts vibrations to the surface of the contact area.

Sound is transmitted to the patient by using the coupling of bone sound into a head bone of the patient instead of transmitting sound through air. In this method the actuator serves as a sound transducer. In contrast with a normal sound transducer, the actuator does not convert an electric signal into air pressure differences, but rather into vibrations that are coupled into the head bone of the patient. In order to achieve the best possible coupling-in, the coupling-in may occur in an area in which the bone is covered by minimal skin and/or tissue. Therefore, the patient may be supported such that the patient contacts the contact area with an area of the frontal bone and/or of the temporal bone, such as in the area of the mastoid process of the temporal bone.

In using bone sound, the sound vibrations through the bone are perceived while bypassing the outer and the middle ear. Therefore, a sound deadening mechanism used in magnetic resonance tomography to suppress noises developed by the gradient coils does not lead to an attenuation of the coupled-in sound. At the same time, with a coupling-in of bone sound, an acoustic perception is achieved that largely corresponds to that which may be perceived when hearing corresponding sound waves. The actuator may thus essentially be actuated with the same signals with which a loudspeaker may be controlled. In such cases, sound may be transmitted by the actuator over the entire range of human hearing, such as, for example, between 2 Hz and 30 kHz, and between 20 Hz and 20 kHz. In such cases, the frequency range typically used by speech may be transmitted, such as the range from 80 Hz to 12 kHz.

The patient communication device may include a microphone for picking up an information signal supplied to the actuator as a control signal by the control device. In some cases, the signal may be exclusively buffered or amplified by the control device. However, a preprocessing of the information signal may also be provided. Preprocessing may include analog-digital/digital-analog conversion, in which a filtering and/or a frequency compensation may be implemented.

As an alternative or in addition to the information signal being picked up via a microphone, the signal may also be digitally stored. In this case, corresponding information is selected through a control input of a user or a device controlling the examination, converted into an information signal and may be supplied to the actuator as a control signal. Such information may involve recorded voice information, however any other stimuli, such as low-frequency vibrations for example, may be transmitted in order to give instructions to the patient.

In this case, the component may be configured as a local coil carrier or a head support. The component may be a carrier of a head coil. A head support in this case provides a support and stabilization function for the head of the patient, such as to prevent a movement of the head during the examination. In such cases, the support may be made of plastic for example or of a sufficiently rigid foam cushion wedge.

The component may however also be configured as a fixing element for fixing the supported patient, for example as webbing or as a headset. The fixing element is disposed on the patient's head and initiates or facilitates a coupling to the head coil. The component may be a sound protection component, such as a sound protection headset that, for example, has additional support surfaces behind the ear that rest on the head surfaces and may be configured to vibrate by an actuator.

In order to provide simple cabling between the actuator and the control device, the magnetic resonance tomograph may include a multi-pin connector for connecting the head coil with a measurement and/or control device assigned to the head coil. The control signal for the actuator is routed through the connector. Connectors are used for easy coupling and uncoupling of local coils to a magnetic resonance tomograph. The connectors may be connected to the head coil via a cable. The connector may be arranged directly on the head coil. In such cases, a connector format may be specified for the connectors in which, when a head coil is used, one or more pins may be unassigned. Therefore one or more of these pins may be used for activating the actuator. In such cases, as well as a pin for a control signal, an additional pin for a ground connection of the actuator may be provided. The actuator and the head coil may also use a common ground. The use of a common connector for the actuator and the head coil on the one hand achieves simple cabling. On the other hand, shared sheath current protection for the measurement and/or control signals of the head coil and for the control signals of the actuator may be used in such cases.

As an alternative to the use of a separate pin, a common signal line may carry both a measurement or control signal of the head coil and also the control signal of the actuator. The common signal line may be used in cases in which the measurement or control signals of the head coil and the control signals of the actuator lie in different (e.g., markedly different) frequency ranges. In such cases measurement signals of a magnetic resonance tomograph typically lie in the megahertz range and audio signals with which the actuator is activated typically lie in the hertz or low kilohertz range. The frequency range of control signals for the head coil depends on the actual application.

For transmission over a common signal line the measurement or control signal of the head coil may be mixed in various ways with the control signal of the actuator. In one case the signals may be summed The signals may be separated by filters. As an alternative, mixing using non-linear mixing processes, for example, frequency or amplitude modulation with corresponding demodulation, may be used.

The actuator may be a piezoelectric actuator. Magnetic components may thus be avoided. In addition, piezoelectric actuators may have relatively small surfaces, such that a low induction of eddy currents in the magnetic resonance tomograph is to be expected. In this case the actuator may be formed from at least one piezoelectric layer disposed on at least one side on a membrane or a housing section of the component. The membrane or the housing section is bendable by a changing shape of the piezoelectric layer. This allows flexural vibrations of the membrane or of the housing section to be created. The excitation of the flexural vibration may be performed in such cases in accordance with the monomorphic or bimorphic principle, in which the piezoelectric layer contracts or expands depending on the control signal, while the expansion of the membrane or of the housing section remains essentially the same. This leads to the bending of the membrane or of the housing section. In order to amplify this effect a piezoelectric layer may be disposed on both sides of the membrane or of the housing section. The two piezoelectric layers are operated with different polarities, such that a control signal that leads to the expansion of one of these layers, leads to contraction of the other layer. Thus the two piezoelectric layers act together in order to create a bending. The use of a piezoelectric layer for bending a membrane or a housing section provides a flat actuator with a large stroke.

Actuators may be used in the contact area to detect information. The actuators may detect (e.g., pick up) vibrations and thus also bone sound caused by the patient speaking. The actuator or a further actuator disposed on the component may serve as a sensor for picking up vibrations as a measurement signal. The control device is configured to control a sound transducer as a function of the measurement signal. Thus a microphone function of the actuator, such as for picking up bone sound, is achieved. In this case the sound transducer may be external, e.g., outside the magnetic resonance tomograph or disposed at a distance from the magnetic resonance tomograph. The control device may be configured such that picking up the measurement signals is only undertaken at times at which no sound output, i.e. no output of control signals, is occurring.

If the separate lines are used for picking up the measurement signal from those used for transmission of the control signals to the actuator, the lines may be routed via the coil connector of a head coil. As described above for the control signals, this may be achieved both via one or more separate pins or also by mixing the signal with a measurement or control signal of the head coil.

A method is also provided for transfer of acoustic information to a patient in a magnetic resonance tomograph. A patient is supported in the magnetic resonance tomograph such that the head of the patient is in mechanical contact with the contact area of a component of the magnetic resonance tomograph. Information is transferred through the component by activation of an actuator by a patient communication device with an electric signal representing the information. In this case the patient may be supported such that his or her head is in contact with the contact area in the area of the frontal bone and/or of the temporal bone, such as in the area of the mastoid process of the temporal bone. The information may be detected (e.g., picked up) acoustically, such as by a microphone. In this case, as described above for the magnetic resonance tomograph, bidirectional communication may be achieved if the actuator is additionally used as a microphone for head sound. The method may be achieved using a magnetic resonance tomograph as described herein.

### BRIEF DESCRIPTION OF THE DRAWINGS

FIG. 1 shows a sectional, partial view of an exemplary embodiment of a magnetic resonance tomograph.

FIG. 2 shows a sectional view of a head coil of an exemplary embodiment of a magnetic resonance tomograph.

FIG. 3 shows schematically the activation of an actuator in an exemplary embodiment of a magnetic resonance tomograph.

### DETAILED DESCRIPTION

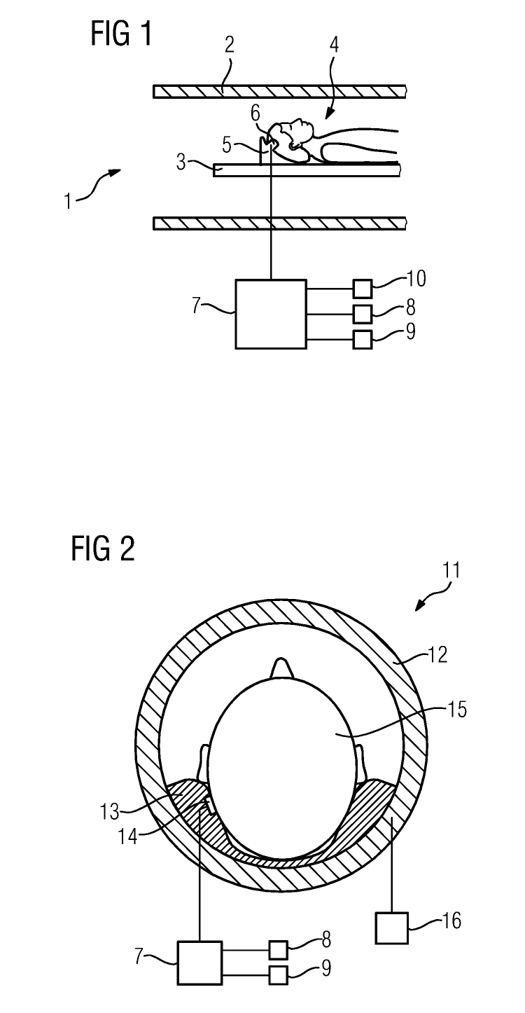

FIG. 1 shows a sectional, partial view of a magnetic resonance tomograph 1. A patient 4 is introduced on a couch 3 into a bore 2 of the magnetic resonance tomograph 1. In this case the head of the patient 4 is supported by a component 5 configured as a head support. A contact area 6 is provided on the component 5, on which the temple of the patient 4 rests during the imaging with the magnetic resonance tomograph 1.

Disposed in the contact area 6 is an actuator not shown in FIG. 1. Upon activation by a control signal, the actuator moves the surface of the contact area 6 facing toward the patient 4. The control signal is provided by the control device 7. The control signal involves an audio signal, such as speech. The patient communication device of the magnetic resonance tomograph 1, in addition to the actuator not shown in the figure and the control device 7, additionally includes a microphone 8 and a loudspeaker 9, which are both disposed outside the bore 2 of the magnetic resonance tomograph. The microphone 8 and the loudspeaker 9 may be at a distance from the bore 2 of the magnetic resonance tomograph, such as disposed in a separate control cabin for example.

Information signals, such as voice inputs of a person, are picked up by the microphone 8. These are supplied by the control device 7 to the actuator. Accordingly the actuator vibrates such that the acoustic information picked up by the microphone 8 is transmitted as bone sound to the skull of the patient 4. The patient thus hears voice inputs that have been made at the microphone 8.

If the patient 4 himself or herself speaks, then the skull of the patient is made to vibrate by the speech of the patient 4, which in turn makes the actuator vibrate. While the actuator is not being controlled by a control signal, the control device 7 may therefore pick up the vibrations of the actuator as a measurement signal and activate the sound transducer 9, which is configured as a loudspeaker, in accordance with this measurement signal, in order to output at the loudspeaker speech of the patient 4 detected by the actuator.

In addition, a control element 10 is also provided on the magnetic resonance tomograph 1, with which an operator may retrieve acoustic information stored in the control device 7. The control element 10 may cause the information to be sent as control signals to the actuator. Thus for example previously recorded speech phrases or acoustic signals that do not represent speech may be reproduced.

FIG. 2 shows a component 11 of a magnetic resonance tomograph. The component 11 is configured as a carrier for a head coil. In this case the component 11 includes a frame 12 that carries the head coil itself and also padding 13, in which the patient's head 15 is accommodated. In this case the actuator 14 is disposed in the padding 13. The actuator 14 is supported by the padding 13 such that the padding 13 is relatively firm in relation to vibrations of the actuator 14 in the audible range, e.g., between 20 Hz and 20 kHz.

The actuator 14 in this case includes a piezoelectric layer disposed on one side on a covering of the padding 13. By activation of the piezoelectric layer with tensions a flexural vibration of the cover may be created which, depending on the control signals of the control device 7, transmits vibrations to the head 15 of the patient. If the actuator 14 is activated with control signals that describe acoustic signals, then the signals are transmitted via the skull of the patient by bone conduction, which makes the audio information audible to the patient.

The further structure of the patient communication device corresponds to the structure in accordance with FIG. 1. An additional measuring and control device 16 is also shown as well in FIG. 2, which detects measurement signals of the head coil.

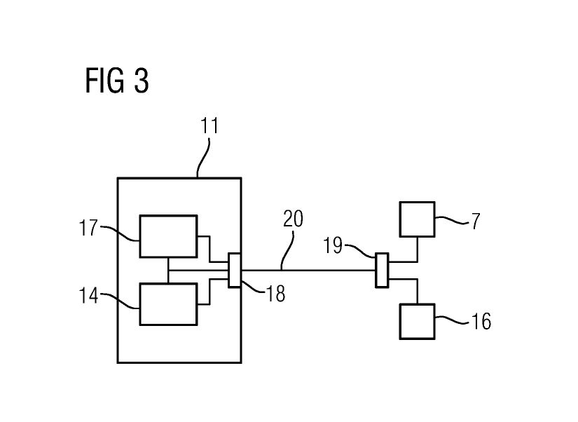

Both in FIG. 1 and also FIG. 2, the signal path of the control signal of the actuator 14 is shown purely schematically. In the case of FIG. 2, the control signal for the actuator may be given via a multi-pin connector for connecting the head coil with the measurement and control device 16 assigned to the head coil 17. For easier understanding this is shown schematically in FIG. 3. The carrier of the head coil, e.g., the component 11, carries the actuator 14 as well as the head coil 17 not shown in FIG. 2 itself. In this case the measurement signals of the head coil 17, control signals for the actuator 14 and also a common ground for both signals are routed to a connector 18 disposed on the carrier of the head coil. The connector 18 is routed via a connection 20 that may be configured as a cable or as conductor tracks in the magnetic resonance tomograph to a further connector 19. The connector 19 has two separate connections that, on the one hand, are connected to the control device 7 of the patient communication system and, on the other hand, are connected to the measurement and control device 16 assigned to the head coil. In this case the control signal of the actuator 14 is routed via a separate pin of the connector 18.

As an alternative, a shared signal line may be used both for the control signal of the actuator 14 and also for the measurement signal of the head coil 17. In this case corresponding filters for signal separation may be provided on the connector 19 and on the connector 18.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A magnetic resonance tomograph comprising:
a component on which a head of a patient rests in a contact area during imaging; and
a patient communication device configured to communicate information to the patient;
wherein the patient communication device comprises a control device and an actuator assigned to the contact area, and
wherein the actuator, upon activation with electric control signals by the control device, vibrates a surface of the contact area.

2. The magnetic resonance tomograph of claim 1, wherein the patient communication device includes a microphone to detect an information signal supplied by the control device to the actuator as a control signal.

3. The magnetic resonance tomograph of claim 1, wherein the component is configured as a carrier of a local coil or as a head support.

4. The magnetic resonance tomograph of claim 3, wherein the component is configured as a carrier of a head coil.

5. The magnetic resonance tomograph of claim 4, further comprising a multi-pin connector to connect the head coil to a measurement device, a control device assigned to the head coil, or both the measurement device and the control device, wherein the control signal to the actuator is routed through the multi-pin connector.

6. The magnetic resonance tomograph of claim 5, wherein a common signal line carries both a measurement signal or a control signal of the head coil and the control signal of the actuator.

7. The magnetic resonance tomograph of claim 1, wherein the actuator is a piezoelectric actuator.

8. The magnetic resonance tomograph of claim 7, wherein the actuator is formed by at least one piezoelectric layer disposed at least on one side on a membrane or a housing section of the component, and wherein the membrane or the housing section is bendable by a changing shape of the piezoelectric layer.

9. The magnetic resonance tomograph of claim 1, wherein the actuator or a further actuator disposed on the component is configured as a sensor to detect vibrations as a measurement signal, wherein the control device is configured to control a sound transducer as a function of the measurement signal.

10. A method for communicating acoustic information to a patient in a magnetic resonance tomograph, the method comprising:
supporting a patient in the magnetic resonance tomograph such that a head of the patient is in mechanical contact with a contact area of a component of the magnetic resonance tomograph; and
communicating information to the patient via activation of an actuator of the magnetic resonance tomograph by a patient communication device with an electric signal representative of the information;
wherein the activation of the actuator with the electric signal vibrates a surface of the contact area.

11. The method of claim 10, further comprising:
detecting an information signal with a microphone; and
supplying the information signal to the actuator as the electric signal.

12. The method of claim 10, wherein the component is configured as a carrier of a local coil or as a head support.

13. The method of claim 10, wherein the component is configured as a carrier of a head coil.

14. The method of claim 13, further comprising connecting the head coil to a measurement device, a control device assigned to the head coil, or both the measurement device and the control device, wherein communicating the information comprises routing the electric signal to the actuator through the multi-pin connector.

15. The method of claim 14, wherein routing the electric signal comprises transmitting both a measurement signal or a control signal of the head coil and the control signal of the actuator on a common signal line.

16. The method of claim 10, wherein the actuator is a piezoelectric actuator.

17. The method of claim 10, further comprising:
detecting vibrations as a measurement signal with the actuator or a further actuator; and
controlling a sound transducer as a function of the measurement signal.
